(19) United States
(12) Patent Application Publication
(10) Pub. No.: US 2014/0221754 A1
(43) Pub. Date: Aug. 7, 2014

(54) SURGICAL LIGHTING ASSEMBLY

(71) Applicants: François Cabaud, Chatillon le Duc (FR); Christophe Favret, Chaux la Lotiére (FR)

(72) Inventors: François Cabaud, Chatillon le Duc (FR); Christophe Favret, Chaux la Lotiére (FR)

(21) Appl. No.: 14/131,188

(22) PCT Filed: Jul. 5, 2012
(86) PCT No.: PCT/IB2012/053451

§ 371 Date: Apr. 10, 2014

(30) Foreign Application Priority Data
Jul. 6, 2011 (FR) 11 56104

(51) Int. Cl. A61B 1/00; A61B 1/06

(52) U.S. Cl. CPC A61B 1/00142; A61B 1/06; A61B 1/0684
USPC 600/178; 600/249

(57) ABSTRACT

The present invention concerns a sterilisable and sterile distributor platform (10) for a surgical lighting assembly, comprising:—at least one base provided with at least one connector (30) capable of ensuring the electrical and mechanical connection of at least one lighting device (1), and—at least one mechanical attachment device capable of attaching the base to a surgical instrument and/or to the sterile operating field.)

The present invention concerns surgical lighting systems.

A surgical operation demands an intensive light close to the area to be treated in order to allow the surgeons to assure their movements.

As is known, operating theatres in hospitals and clinics are equipped with powerful lighting systems, predominantly fixed to the ceiling, providing a directional beam from top to bottom. The use of a headband lamp is likewise known, this often being uncomfortable and requiring the surgeon to position his head on the axis of his sight.

Flesh, which is naturally red, has a tendency to absorb the visible spectrum.

Additional lighting fixed in proximity to the patient, close to the area to be operated on, affords work comfort and allows both the movement of the surgeon to be reassured and time to be gained in the operation.

The lighting system must not emit too much heat, however, so as not to risk burning the flesh of the patient if it were to come into contact with the lighting system and must not emit heat that can hamper the surgeon.

The application US 2005/0171407 relates to a lighting system having one or more light emitting diodes (LED) carried by a semiflexible stem that is formed by more than ten or so elements fitted into one another. The lighting system can be fixed to different locations such as the ceiling, wall or floor.

The Japanese application JP 2007/282920 describes a lighting system that can be installed on an operating table and that has a plurality of articulated arms that are each terminated by a light source, for example an LED diode.

The application DE 2344794 discloses the practice of using optical fibers to light an operating area. U.S. Pat. No. 5,722,426 discloses a supple light probe having a bundle of optical fibers for internally lighting the blood vessels, and WO 02 103 409 discloses an optical system for guiding a catheter using an optical fiber and a laser diode.

The application WO 02/07632 discloses a plurality of lighting devices, which can be used in medicine or in surgery, which are battery powered and have at least one LED diode.

There is a need to further improve existing lighting systems and make the use thereof more ergonomic and better suited to various operating situations.

According to a first of its aspects, the invention relates to a sterilizable and sterile distributor cap, for a surgical lighting assembly, having:
a base having at least one connector capable of electrically and mechanically connecting a lighting device, and
at least one mechanical attachment device capable of attaching the base to at least one surgical instrument and/or to the sterile operating field.

The invention makes it possible to use one and the same base and one or more lighting devices that may differ from one operation to another, on the basis of the lighting required.

In particular, the base advantageously has a plurality of connectors and the lighting devices may have a flexible or semiflexible stem, according to the operating situations.

The lighting device may be provided for single use on account of its being separable from the base.

The invention also relates to the lighting device as such.

Distributor Cap

The connector(s) of the cap is/are preferably of female type, which facilitates the safety of the patient and allows the size of the lighting device to be limited.

The distributor cap is designed to be washable and sterilizable, for example using a vapor phase autoclave.

The distributor cap receives the current from a power supply and/or a storage battery via a cable and distributes it to the lighting device(s) via the connector(s).

The power supply may be of any type known to a person skilled in the art. In particular, the base may be connected to the mains via a transformer or a switching power supply. The base may have a current regulator. In one variant, the base may have a sterilizable storage battery. Preferably, the power supply is a low voltage power supply. The power supply may comply with the TBT label in ISO standard 60601-1.

The power supply does not need to be sterilized, but nevertheless can be.

The distributor cap may have various shapes, depending on the support to which it needs to be attached.

The distributor cap can be attached to an operating table, to cover a sterile operating field. The distributor cap can be fixed to a tube, trocar or other surgical tool.

The invention also relates to a surgical lighting assembly having:
a distributor cap as defined above, and
at least one sterilizable and sterile lighting device, notably for single use, having:
   a connector electrically and mechanically connecting to a corresponding connector on the distributor cap,
   at least one light source, preferably at least one LED diode.

Lighting Device

The lighting device may be designed to be introduced into an orifice, either natural or unnatural, in the human body, without hampering manipulations by the surgeon.

The lighting device may have a flexible or semiflexible stem.

The connector situated at the proximal end of the stem allows the latter to be mechanically fixed into the distributor cap. It is preferably a male connector.

The device may be for single use. In one variant, the device is washable and sterilizable a plurality of times.

The lighting device may be designed to have a color temperature greater than 4500 K (kelvin), notably greater than 6700 K, or greater than 10 000 K, or even greater than 15 000 K, in stable operation. A high color temperature, corresponding to a blue light, is of particular benefit for lighting red flesh. The light source may have one or more LED diodes, for example an LED matrix.

The number of LED diodes is preferably between 1 and 10.

In one particular embodiment, the light source has one or more LED diodes that are chosen so that the color temperature of the lighting device in stable operation is greater than 15 000 kelvin. The choice of LEDs is made by means of qualitative selection from a batch of LEDs, for example a batch of white LEDs, on the basis of the performance of each LED as shown on a Gaussian curve. Only the better part of the production is retained.

In order to obtain the desired color temperature, the diodes are supplied with a current of between 50 mA and 100 mA, in particular between 70 mA and 90 mA, or close or equal to 80 mA.

By way of example, LED diodes supplied by NICHIA under the reference: NSPW500GS-K1 are used. Through “Gaussian” screening, only the best performing LEDs from the batch are kept, having a maximum luminosity of 44 000 mcd, whereas the average for the batch is generally around 33 000 mcd.

By way of example, the diodes are supplied with a current at a level between 20 mA and 30 mA; the lighting is then bluish and the temperature is then greater than 10 000 K.

In another example of use, the diodes are supplied with a current of level 80 mA, ie four times the recommended level, which produces lighting that draws even closer to blue and a temperature greater than 15 000 K.

More generally, the diodes are supplied with a current of between 10 mA and 100 mA, particularly between 10 mA and 35 mA or between 50 mA and 90 mA, according to the desired use. By way of example, a level of between 10 mA and 35 mA, or around 20 mA, is of benefit to the introduction of the device into the body and the lighting of a confined operating field that does not require intensive lighting and where it is particularly important to limit the risks of overheating. A higher level, for example greater than or equal to 60 mA, or 70 mA or even 80 mA, is advantageous in order to obtain a high color temperature, as seen previously, for example in order to light flesh using a device fixed outside the body.

The light source may belong to a light head situated at the distal end of the lighting device, forming a directional spot or, in one variant, multidirectional lighting.

The presence of a directional or multidirectional light head is advantageous when the stem if semiflexible.

The lighting device may have a thermistor in order to detect an excessive temperature for the light source, and the lighting assembly may have a safety device that signals the excessive temperature, interrupts the operation of the lighting device or, preferably, reduces the level thereof in the event of a temperature above a predefined threshold.

In a preferred embodiment, the thermistor is soldered directly to the negative part, which corresponds to the hottest part, of an LED in the light head. This singular configuration allows a very fast response to the temperature rise.

The thermistor is embedded in a resin, for example.

The thermistor is, by way of example, chosen to lower the current level from 80 mA to 20 mA in the event of a threshold temperature of 40° C. being exceeded.

The thermistor can be replaced with a component that interrupts or limits the flow of current in the event of a temperature above a predefined threshold.

The light head may be removable or permanently fixed to the lighting device.

The lighting device, particularly the light head, may be at least partially coated with glue, so as to allow it to be fixed as chosen by the user. The glue used may be a repositionable glue, which is acceptable in a surgical environment, notably nontoxic. By way of example, the glue is a liquid glue deposited on the stem during factory production of the device, notably in the form of a lace parallel to the axis of the stem. In order to protect it during transport and storage, the glue may have been covered, following a drying time, with a film of PTFE that the surgeon peels off before sticking the stem in the desired position.

By way of example, the adhesive part produced in this manner can allow the light head to be stuck and peeled off at least 10 times on a plastic or metal support or on healthy skin in accordance with an ISO standard 10993.

In one implementation example, the stem is semiflexible and moldable. A “semiflexible” stem is intended to be understood to mean a semi rigid stem that is capable of preserving the configuration in which it is left by the user. The stem is thus sufficiently rigid to keep its shape once left in the configuration sought by the user. The stem can thus take an appropriate shape, under the action of the surgeon. The malleable nature of the stem allows the surgeon to position the light beam at his convenience, in particular inside the body being operated on.

The light beam emitted by the light head that is integral with the semiflexible stem is preferably oriented on the axis of the distal part of the stem.

The semiflexible stem may have an electric cable, for example a copper or aluminum monofilament wire surrounded by an insulating layer, particularly made of PTFE or made of another plastic material, and a braided metal frameworkwork, particularly embedded in a ductile metal that provides the assembly with its malleable nature. Any ductile metal, such as tin or aluminum, can be used, and the optional presence of an external silicone layer allows any contact between the frameworkwork and the patient or the practitioner to be avoided. The metal core conducts one pole, and the framework conducts the other pole.

In another implementation example, the stem is flexible. A “flexible stem” is intended to be understood to mean a supple stem that is capable of losing the configuration in which it is left by the user under the effect of its own weight.

When the stem is flexible, it may have a light head at its distal end and/or a plurality of LED diodes distributed over at least part of the length of the stem, thus allowing a peripheral lighting area to be created.

Depending on the position given to the part of the stem having the diodes, all the LED diodes can be directed into one and the same direction, notably into a direction perpendicular to the longitudinal axis of the stem.

The distal end of the lighting device, notably in the case of a flexible stem, can be at least temporarily stiffened in order to facilitate the introduction thereof into an orifice, particularly an orifice in the human body. The human body is naturally opaque, and a supply of light internally is particularly advantageous for visualizing an operating area.

The stiffening can be obtained by using a rigid tube into which the lighting device is introduced, but it can also be obtained by the addition of a metal rod that is temporarily introduced into a longitudinal light from the stem.

The setup of the lighting device, notably with a flexible stem, in a human body can be implemented using a large needle, a trocar or an introduction means provided for this purpose, for example.

The length of the flexible or semiflexible stem varies according to use.

The length of the stem may be between 40 cm and 120 cm, preferably between 40 cm and 80 cm.

In the case of a flexible stem, the length of the portion that includes the LED diodes is between 1 and 30 cm, for example.

The greatest transverse dimension of the stem is preferably less than or equal to 3 mm.

The invention will be able to be better understood upon reading the detailed description that follows, of nonlimiting implementation examples therefor, and upon consideration of the appended drawing, in which:

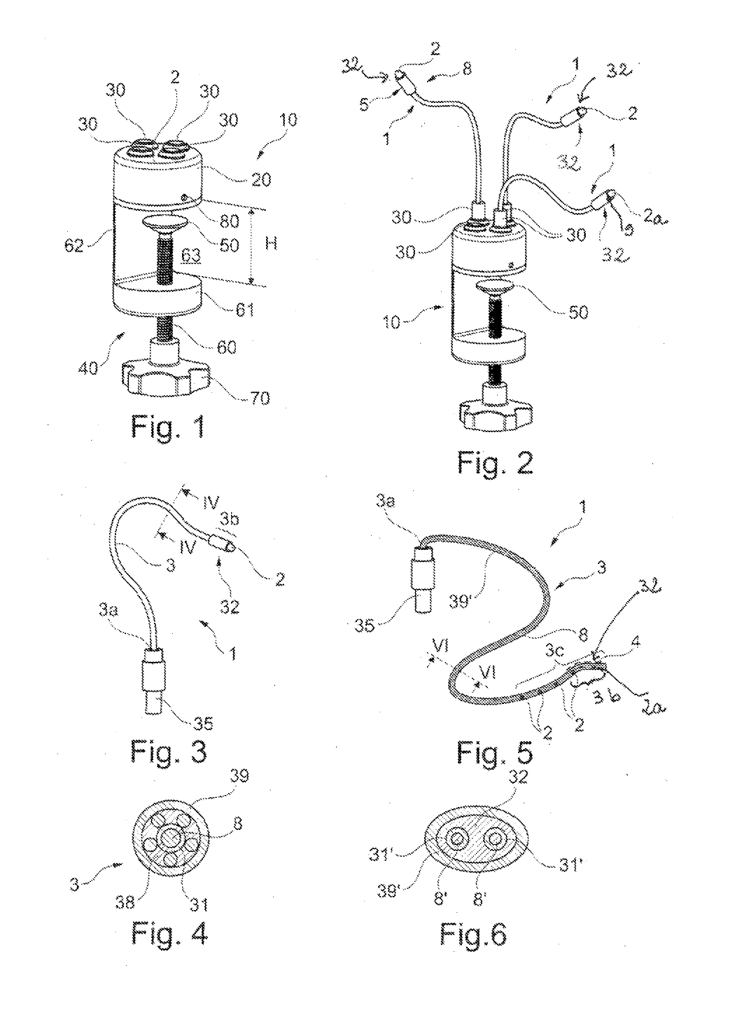

FIG. 1 shows a perspective view of a distributor cap produced in accordance with the invention,

FIG. 2 shows the cap from FIG. 1, equipped with three lighting devices having a semiflexible stem,

FIG. 3 shows a lighting device with a semiflexible stem in isolation,

FIG. 4 shows a section along IV-IV through the lighting device from FIG. 3,

FIG. 5 shows a lighting device with a supple stem in isolation,

FIG. 6 shows a section along VI-VI through the lighting device from FIG. 5, and

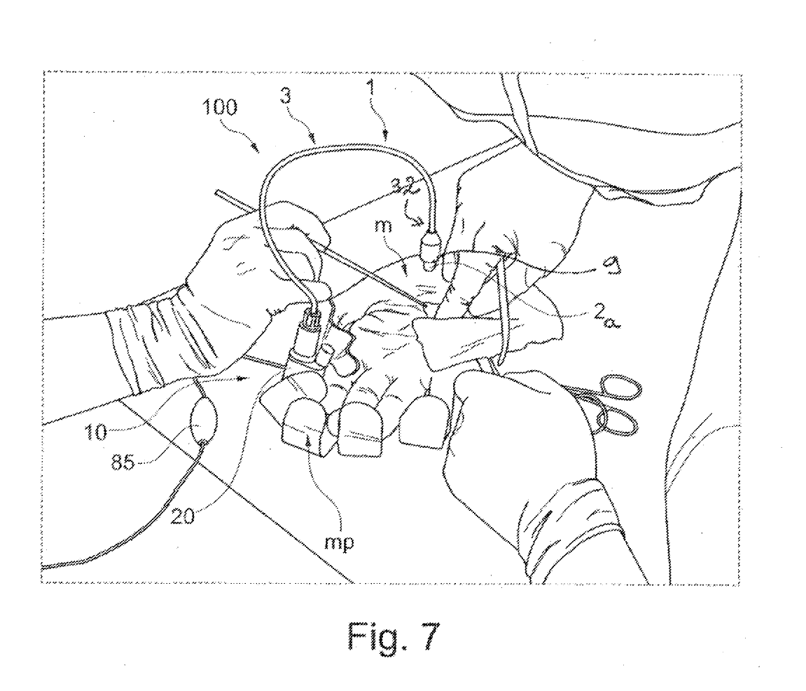

FIG. 7 shows an example of use of a surgical lighting assembly according to the invention.

The distributor cap 10 illustrated in FIG. 1 has an aluminum base 20 fitted with connectors 30 and a fixing means 40, which is suited to fixing to a table, a trocar or another surgical instrument, for example.

In the example under consideration, the base 20 is fitted with four connectors 30, each both mechanically holding and electrically connecting an associated lighting device. In the exemplary embodiment illustrated, these connectors 30 are female connectors that are present on the upper face 21 of the base 20.

The base 20 likewise has a socket 80 allowing it to be connected by a chord to a power supply, which is not shown. By way of example, the socket 80 is present in the lower part of the base 20, and it is preferably a female socket.

In the example illustrated, the fixing means 40 is of clamp type, and has a stainless steel clamping screw 60 that can be adjusted using a knurled wheel 70, the screw 60 being screwed into a foot 61 that extends under the base 20 and that is connected thereto by an upright 62. That end of the screw 60 that is opposite the base 20 is fitted with a bearing cup 50, which can be articulated on the screw 60.

The interval 63 between the foot 61 and the base 20 has a height H of between 10 and 30 mm, for example.

There is no departure from the scope of the invention when the base is equipped with another type of fixing, which may take different forms depending on the instruments or the areas on which the cap needs to be fixed. The embodiment illustrated in FIG. 1 is suited quite particularly to fixing to the operating field. In a variant that is not shown, the fixing means may have a spring clamp. The connectors are the same whatever the base.

The distributor cap 10 is sterilizable.

FIG. 2 shows the distributor cap from FIG. 1, fitted with lighting devices 1 having semiflexible stems.

One of the connectors 30 is not used, for example, as illustrated.

The distal end 3b of one of the lighting devices 1 has a thermistor 9 that is soldered to the negative part of an LED 2a.

The lighting device 1 having a semiflexible stem 3 that is illustrated in FIG. 3 has a male connector 35 that is situated at the proximal end 3a of the stem 3 and a light head 32 having an LED diode 2, which is situated in the distal part 3b of the same stem.

In this embodiment, the semiflexible stem has—as illustrated in FIG. 4—a copper core 8 covered with a PTFE sheath 31 and with a braided copper framework 38 embedded in the tin. To ensure biocompatibility, the stem is moreover covered with an outside layer 39 in the form of a silicone casing.

The lighting device 1 having a flexible stem 3 that is illustrated in FIG. 5 has a supple stem 3 that is formed by a silicone casing 39′, containing two copper wires 8′ in a PTFE sheath 31′ for the supply of power to a plurality of LED diodes 2, in this case numbering 5, which are arranged at regular intervals along part 3c of the length of the stem as from the distal end thereof. FIG. 6 moreover shows a silicone filling 32; following reticulation, the silicone thus injected into the casing 39′ allows the LEDs to be held and the electrical connections to be reinforced while keeping the suppleness for the stem 3.

The stem 3 shown in section in FIG. 6 has a flattened transverse section, whereas the stem in FIG. 4 had a circular transverse section; the invention is not limited to semiflexible or flexible stems having a transverse section of a particular shape. The stem of a lighting device according to the invention may have a transverse section that is circular, oval, polygonal, notably triangular, square, rectangular, octagonal, parallelogrammatic or rhombic.

In a variant that is not illustrated, the distal part 3d of the stem 3 has a rigid tube around the LEDs 2, which facilitates particularly the insertion of the lighting device in the case of internal use.

The distal part of the lighting device 1 illustrated has, moreover, an adhesive area 4, for example coated with a repositionable glue, that allows the lighting device 1 to be fixed to a surgical instrument or to the skin of the patient. The LEDs diodes 2 can then be positioned on a preferred quadrant opposite the stickable face.

In another variant that is not illustrated, the stem is not treated with a repositionable glue and the LED diodes are distributed in order to light 360° around the stem.

FIG. 7 illustrates the use of a surgical lighting assembly 100 during an operation on the hand. The fingers of the hand m are held out of the way by a lead hand mp on which the support 10 of the assembly 100 is fixed.

In other operations, the cap may be fixed on an abdominal surgical retractor, for example, or the side rails of the operating tables.

In the embodiment in FIG. 7, the assembly has a single lighting device 1, the single LED 2a providing satisfactory lighting. By way of example, said LED is supplied with a current at a level equal to 80 mA, and the color temperature is greater than 15 000 K. A thermistor 9 is connected directly to the LED 2a in order to limit the supply current in the event of a threshold temperature being exceeded by the LED.

Advantageously, the base 20 has small dimensions, particularly since it does not have a current regulator. The base is supplied with power via a separate current regulator 85 that is embedded in a small silicone coating.

The invention is not limited to the examples illustrated.

By way of example, the distributor cap may have fixing means that are different than those illustrated, as mentioned previously.

The number of connectors that are present on the cap may be different, for example ranging from one to ten. According to the operation envisaged, the practitioner chooses to use one or more lighting assemblies, notably a plurality of assemblies with a cap of small size designed for a single lighting device or a cap having a plurality of connections.

The light sources preferably provide lighting in white light, but other colors can be used.

Each lighting device 1, having a flexible or semiflexible stem, can be packaged in sterile fashion in a medical bag.

The distributor cap may be offered separately, likewise in sterile form in a medical bag.

The cap may have a dimmer allowing the light level to be controlled if need be.

The invention is not limited to a particular type of operation. Among the possible uses, apart from surgery on limbs, it is possible to cite lighting for proctology and gynecology, this list not being exhaustive.

The expression “having a” is intended to be understood to be synonymous for “having at least one”.

1. A sterilizable and sterile distributor cap, for a surgical lighting assembly, having:
at least one base provided with at least one connector capable of electrically and mechanically connecting at least one lighting device designed to attain a color temperature greater than 10,000 kelvin, in stable operation, and
at least one mechanical attachment device capable of attaching the base to a surgical instrument and/or to the sterile operating field.

2. The distributor cap as claimed in claim 1, the connector being a female connector.

3. The cap as claimed in claim 1, having at least three, preferably four, connectors.

4. The cap as claimed in claim 1, having a single connector.

5. A surgical lighting assembly, having:
a distributor cap as claimed in claim 1,
at least one lighting device, notably for single use, having:
a connector electrically and mechanically connecting to a corresponding connector on the base of the distributor cap,
at least one light source comprising at least one LED designed to attain a color temperature greater than 10,000 kelvin in stable operation.

6. The assembly as claimed in claim 5, the lighting device having a semiflexible stem.

7. The assembly as claimed in claim 6, the stem having an electric cable surrounded by a braided metal framework, embedded in a ductile metal.

8. The assembly as claimed in claim 5, the lighting device having a flexible stem, notably having an envelope made of a transparent thermoplastic material.

9. The assembly as claimed in claim 8, having a plurality of LED diodes distributed along a portion of the length of the stem.

10. The assembly as claimed in claim 5, the lighting device being at least partially coated with a repositionable glue.

11. (canceled)

12. The assembly as claimed in claim 5, the lighting device being designed to attain a color temperature greater than 15,000 kelvin in stable operation.

13. The assembly as claimed in claim 5, the lighting device having a thermistor.

14. The assembly as claimed in claim 13, a distal end of the lighting device having a light head having an LED, the thermistor being soldered to the negative part of said LED.

15. The assembly as claimed in claim 5, the lighting device being designed to be introduced into an orifice, either natural or unnatural, in the human body.

16. A sterile lighting device, for an assembly as claimed in claim 5.

17. The assembly as claimed in claim 5, the lighting device being supplied with a current of between 10 mA and 100 mA.

18. The assembly as claimed in claim 17, the lighting device being supplied with a current of between 10 mA and 35 mA.

19. The assembly as claimed in claim 17, the lighting device being supplied with a current of between 50 mA and 100 mA.
